(19) United States
(12) Patent Application Publication
(10) Pub. No.: US 2026/0345315 A1
(43) Pub. Date: Nov. 5, 2026

(54) System, Device and Method for Repairing a Rotator Cuff Tear

(71) Applicant: KiriGenX, Inc., Phoenix, AZ (US)

(72) Inventors: Theodore R. Kucklick, Scotts Valley, CA (US); Louis McIntyre, Winston Salem, NC (US); Robert T. Burks, Salt Lake City, UT (US)

(73) Assignee: KiriGenX, Inc., Phoenix, AZ (US)

(21) Appl. No.: 19/745,555

(22) Filed: Jul. 17, 2026

Related U.S. Application Data
(63) Continuation of application No. 19/024,100, filed on Jan. 16, 2025, now Pat. No. 12,685,629.
(60) Provisional application No. 63/624,601, filed on Jan. 24, 2024.

(51) Int. Cl. A61F 2/08; A61B 17/00

(52) U.S. Cl. CPC A61F 2/0811; A61B 17/00234; A61B 2017/00238

(57) ABSTRACT

A delivery device used to deploy a biological construct into the glenohumeral joint space for minimal disruption to repair a partial rotator cuff tear. The biological construct is positioned on the articular side of the tendon tear and introduced into the joint space so that the construct supports the tear without further tearing or damaging the tendon. The patch is secured to the tear with stiches on the bursal side of the tear.

This application is a continuation application of U.S. Application 19/024,100, filed Jan. 16, 2025, which claims priority to U.S. Provisional Application 63/624,601, filed Jan. 24, 2024.

### FIELD OF THE INVENTIONS

The inventions described below relate to the field of systems and methods of arthroscopic repair of torn shoulder tendons.

### BACKGROUND OF THE INVENTIONS

Rotator cuff tears are a prevalent condition, particularly among athletes and the aging population. It has been reported that many people over the age of 50 have a partial rotator cuff tear with 70% of those over 70 with a partial tear. This represents about 47 million people in the U.S. and 1.2 billion people worldwide. Only 30% of those tears are on the bursal side, and 70% are on the articular side. Without treatment, these partial tears can progress to full thickness tears.

Conventional surgery repair techniques include arthroscopic repair of the partial tendon by completion of the partial tear in order to access the articular side of the tear. While these methods have demonstrated success in restoring function, they present challenges in the healing process and a significant increase in morbidity rates.

There remains a need for more effective and less invasive methods that improve healing times and provide superior outcomes and better recovery, especially for partial tears on the previously inaccessible articular side of an intact rotator cuff. The devices and methods below optimize tendon repair, promote biological healing and minimize the risk of post operative complications resulting in improved functional recovery.

### SUMMARY

The devices and methods described below provide for improved access with minimal disruption to repair a partial rotator cuff tear. A delivery device is used to deploy a regenerative biological construct or patch into the glenohumeral joint space. The construct is positioned on the articular side of the tendon tear and introduced into the joint space so that the construct supports the tear without further tearing or damaging the tendon. The construct is secured to the tear with stiches on the bursal side of the tear. A delivery cone is inserted into an anterior cannula for insertion of the biological construct or patch onto the articular or bottom side of the tendon tear. The biological construct has suture tails on an end of the construct. The construct is compressed to the articular side of the rotator cuff with the sutures and tensioned with the sutures on the bursal side of the tear without requiring a full or completed tear of the torn tendon. The sutures are then tied to the bursal side of the tear with the construct compressed to the articular side of the tear. Once the construct is delivered to the repair site, the suture is then pulled to the tear and tensioned against the construct to secure the construct against the articular side tear of the rotator cuff.

### BRIEF DESCRIPTION OF THE DRAWINGS

FIG. 1 illustrates the significant shoulder anatomy of a patient shoulder including the glenohumeral and subacromial space.

FIG. 2 illustrates the method for placement of a biological construct or patch installed on the articular side or bottom side of the tear of a shoulder tendon of a patient.

FIG. 3 illustrates the method for placement of a biological construct or patch installed on the articular side or bottom side of the tear of a shoulder tendon of a patient with a rolled construct loaded into a roller cone.

FIG. 4 illustrates the view of the glenohumeral space and the articular sided tear with the spinal needle and anterior cannula inserted.

FIG. 5 illustrates The traction sutures are grasped and pulled through the anterior portal through the cannula in the rotator interval.

FIG. 6 illustrates the construct advanced through the glenohumeral joint space and advanced toward the articular sided tear.

FIG. 7 illustrates the construct placed over the articular sided tear.

FIG. 8 illustrates the knotted sutures on the bursal side after placement of the construct against the articular side tear.

### DETAILED DESCRIPTION OF THE INVENTIONS

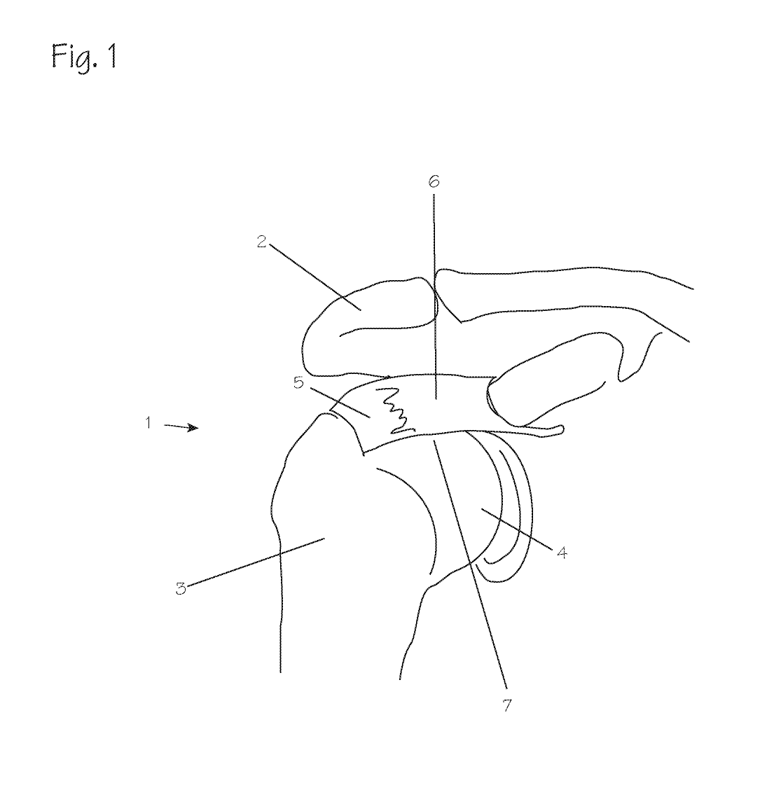

FIG. 1 illustrates the significant shoulder anatomy of a patient shoulder 1 including the glenohumeral and subacromial space. The figure illustrates the acromion 2, humerus 3 glenohumeral joint 4 and supraspinatus 5 of the shoulder. The figure shows a rotator cuff having a bursal or top side of the rotator cuff tear 6 and an articular or bottom side of the rotator cuff tear 7.

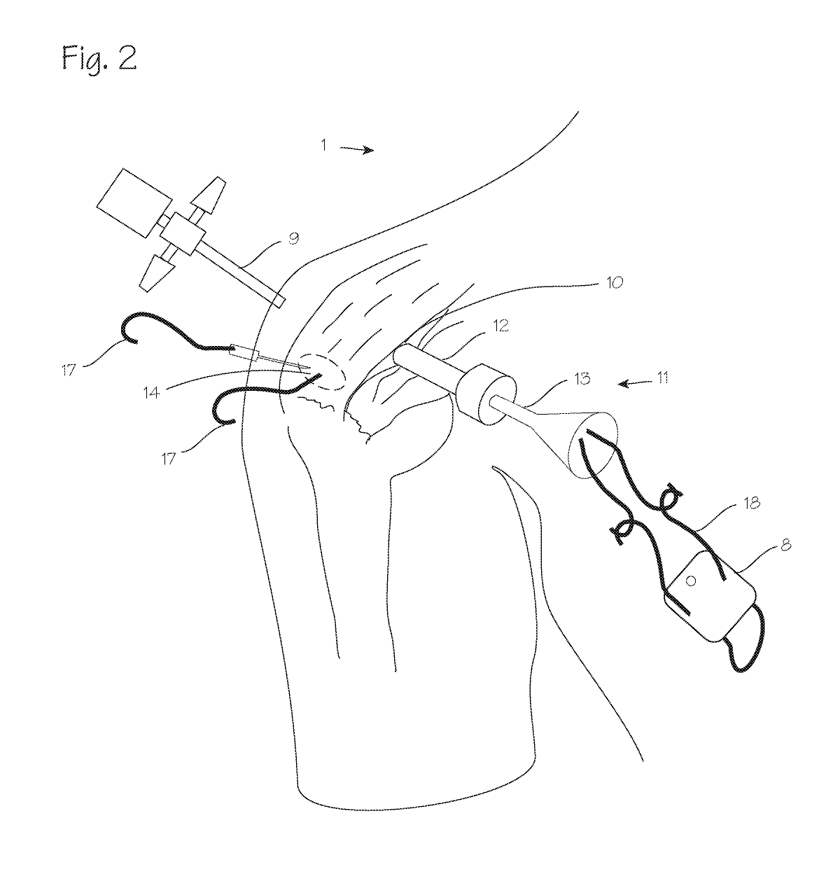

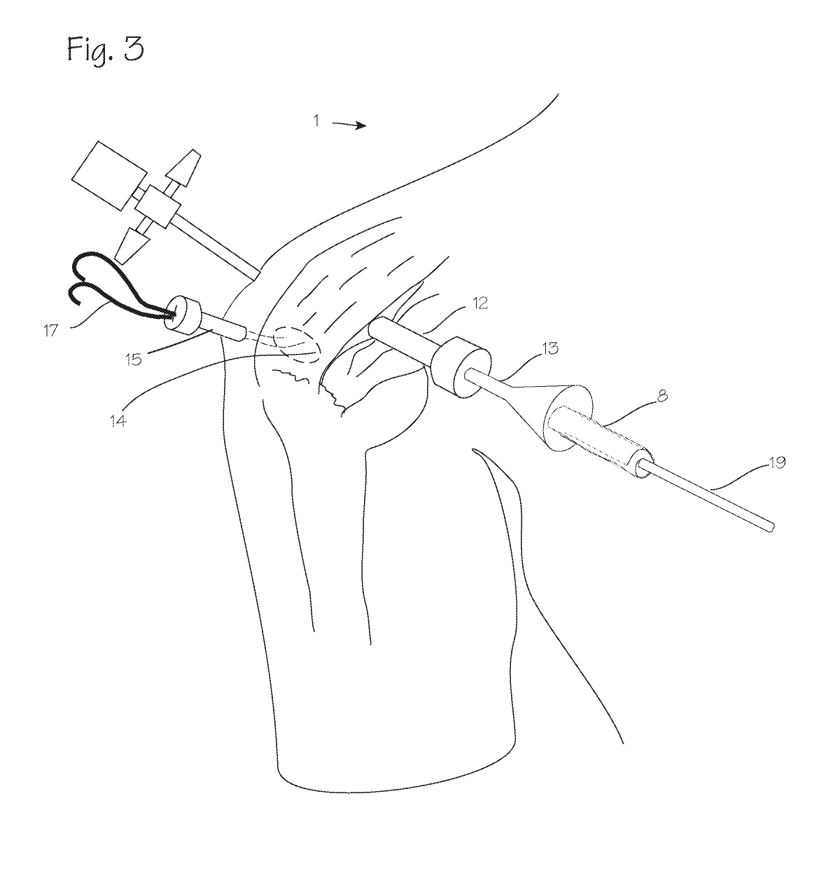

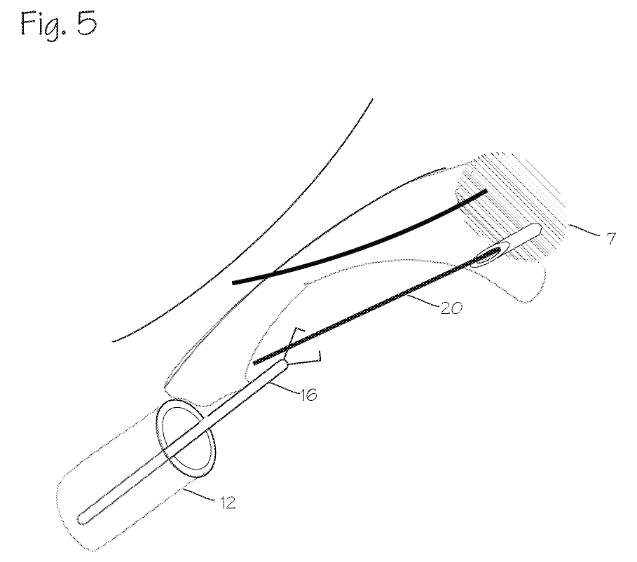

FIG. 2 illustrates the method for placement of a biological construct or patch 8 installed on the articular side or bottom side of a patient with a torn shoulder tendon. First, a viewing portal is established through a posterior portal of the shoulder. The viewing portal can be distended for introduction of a scope 9 for viewing. Then, a first surgical opening 10 is established in the shoulder to create an arthroscopic or anterior portal. The first surgical opening establishes the arthroscopic or anterior portal through the rotator cuff interval. The arthroscopic or anterior portal could be any anterior portal accessing the glenohumeral space. The arthroscopic or anterior portal can include a delivery device 11 inserted therethrough for insertion of sutures and tools. The delivery device can further include an anterior cannula 12 and roller cone 13. The anterior cannula is then inserted into the arthroscopic or anterior portal. A second surgical opening 14 is then established to create a lateral portal and introduce a lateral cannula 15 (shown in FIG. 3). The lateral portal can accommodate a grasper 16 (shown in FIG. 5). After the anterior cannula is inserted, two or more traction sutures 17 are pushed through a spinal needle from the lateral portal, passed through the rotator cuff and pulled out of the anterior cannula via the grasper (shown in FIG. 5). The traction sutures can be Polydioxanone (PDS) traction sutures. The traction sutures are then pulled out of the anterior portal cannula with the grasper. The PDS sutures serve to retract tissue to improve visibility and access during the procedure. The delivery roller cone 13 is inserted into the anterior cannula 12 for insertion of a biological construct or patch onto the articular or bottom side of the tendon tear 7. The biological construct has suture tails 18 on an end of the construct. The traction sutures 15 are tied to the suture tails 18 of the biological construct.

Outside the delivery device, the construct is in a first unrolled position, then inserted through the delivery device in a second rolled position (shown in FIG. 3), and then advanced through the delivery device and expanded back into an open or unrolled position. The construct is advanced through the delivery device and unrolled when positioned in the glenohumeral joint space of the shoulder. A driver or inserter 19 (shown in FIG. 3) may be used to push the biological construct to the glenohumeral joint space while the traction sutures are pulled on at the same time. The construct exits the distal end of the delivery device and is positioned on the articular side or bottom side of the tear. The construct is attached to a suture tail long enough to follow the construct to the tendon repair site at a first end and also trail to the top of the tendon surface at the second end. The biological construct is pulled against the articular sided tear with the traction sutures. The traction sutures pull the surgical sutures through the rotator cuff. The construct suture tails are then pulled out of the lateral cannula and clipped off. The sutures are then tied to the bursal side of the rotator cuff with the implant compressed to the articular side of the rotator cuff. Once the patch is delivered to the repair site, the suture is then pulled to the tear and tensioned against the compressed patch to secure the patch against the articular side tear of the rotator cuff. To tension the patch, the suture is pulled from the first end to oppose the patch to the tendon tear. The second suture end that is on the bursal or top side is also tightened or pulled and secured to the tendon or bone to ensure secure placement of the patch. A knot is tied in the surgical suture, pushed through the lateral cannula and the knot is tied down to the bursal surface while excess surgical suture is clipped off. FIG. 8 illustrates the suture ties on the bursal side of the tendon being optionally secured to a pledget, sponge, pack or plate or anything absorbent to help manage fluids and hold a bioactive substance. The pledget is applied to bursal side to provide a means to cinch the sutures.

FIG. 3 illustrates the method for placement of a biological construct or patch installed on the articular side or bottom side of the tear of a shoulder tendon of a patient with a rolled construct loaded into the roller cone. The delivery cone 14 is inserted into the anterior portal 10 for insertion of the biological construct onto the articular or bottom side of the tendon tear. The figure illustrates the construct after the traction sutures have been tied to the construct suture tails have been tied to the suture ties and rolled from a first unrolled position to a second rolled position and loaded into the roller cone. The construct is then advanced through the delivery device and expanded back into an open or unrolled position. The construct is advanced through the delivery device and unrolled when positioned in the glenohumeral joint space of the shoulder. The driver 19 may be used to push the biological construct to the glenohumeral joint space. The construct exits the distal end of the delivery device and is positioned on the articular side or bottom side of the tear. Once the construct is delivered to the repair site, the suture is then pulled to the tear and tensioned against the compressed patch to secure the patch against the articular side tear of the rotator cuff.

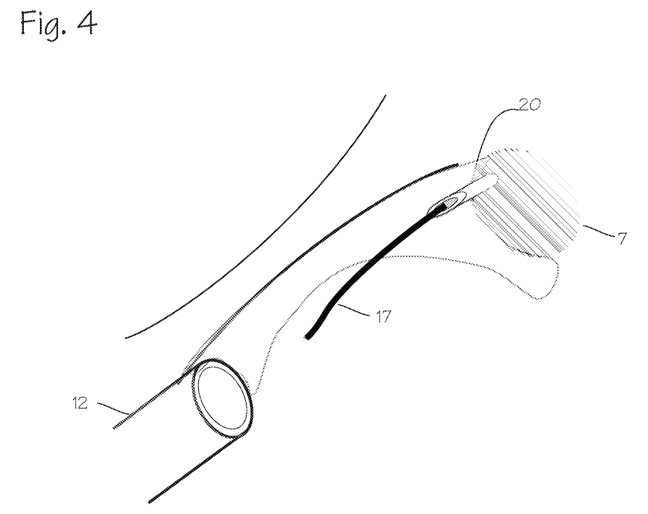

FIGS. 4 through 8 illustrate path of the construct as it is positioned onto the articular sided tear. FIG. 4 illustrates the view of the glenohumeral space and the articular sided tear with the spinal needle and anterior cannula inserted through the articular sided tear. This figure illustrates the arthroscopic view 10 into the glenohumeral space, viewing the articular sided tear. Access to the space can be achieved by advancement of a spinal needle 20 through the articular sided tear 7. The traction sutures are passed into the glenohumeral space through rotator cuff through the spinal needle. The sutures can be retrieved through the cannula in the anterior portal through the rotator interval.

FIG. 5 illustrates the construct being pulled to the articular side tear via the traction sutures. The construct is advanced toward the articular side tear without completion of the tear from the bursal side. The partial tear is maintained as a partial tear and not completed into a total tear. The regenerative patch is applied directly to the tear on the articular side for optimal healing.

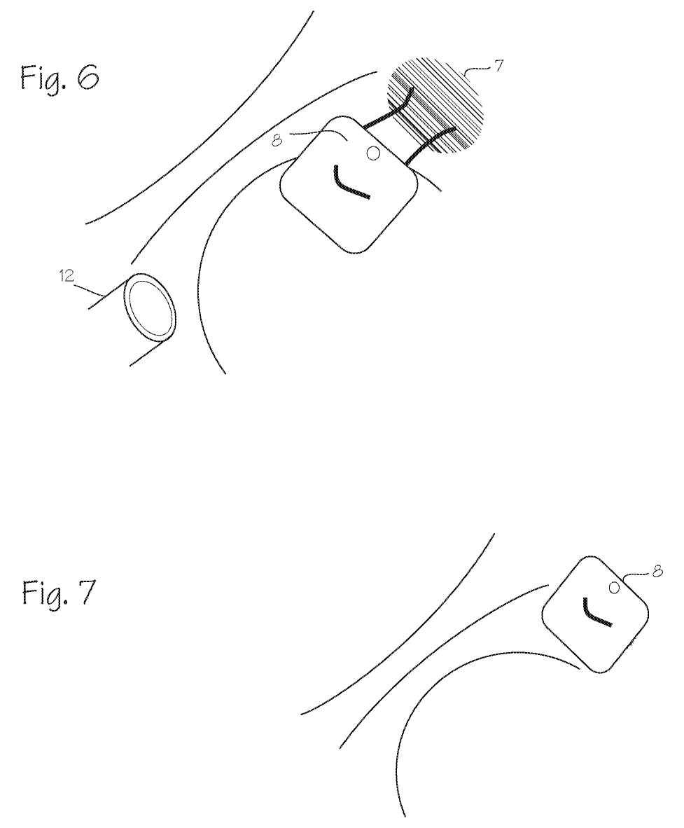

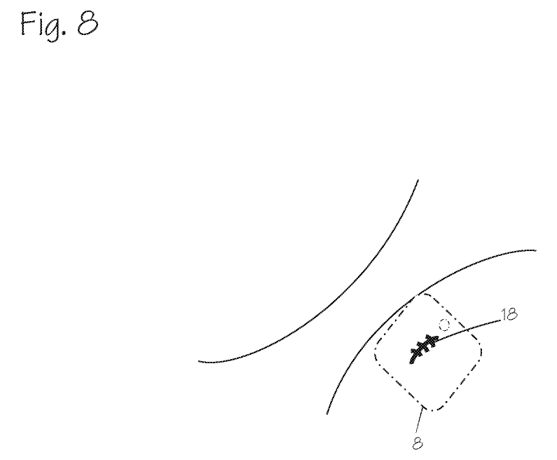

FIG. 6 illustrates the construct 8 advanced through the glenohumeral joint space and advanced toward the articular sided tear 7. FIG. 7 illustrates the construct 8 placed over the articular sided tear. FIG. 8 illustrates the knotted sutures on the bursal side after placement of the construct against the articular side tear.

The rolled construct emerges in a rolled position from the anterior cannula. FIG. 6 illustrates the construct once it is completely out of the anterior cannula, in the glenohumeral joint space as is advances toward the articular sided tear. FIG. 7 illustrates that the construct is attached to a suture string long enough to follow the construct to the tendon repair site at a first end and also trail to the top of the tendon surface at the second end. This provides access and placement of the repair patch to the articular side through the tendon. FIG. 7 illustrates once the patch is delivered to the articular tear site, the sutures are passed through the rotator cuff to lay the patch against the tear. FIG. 8 illustrates the sutures are then pulled to the tear and tensioned against the compressed construct to secure the construct against the articular side tear of the rotator cuff. To tension the construct, the suture is pulled from the first end to oppose the construct to the tendon tear. The second suture end that is on the bursal side is also tightened or pulled to the bone to ensure secure placement of the construct. FIG. 8 illustrates the sutures tied on the bursal side of the tendon. The sutures can optionally be secured to a pledget, sponge, pack or plate or anything absorbent to help manage fluids and hold a bioactive substance.

The construct may alternatively be inserted through the roller cone in the anterior portal and the suture tails retrieved with a small diameter penetrating suture grasper through the rotator cuff tendon, without the use of traction sutures, and the sutures tied on to the bursal side of the rotator cuff to compress the construct against the articular sided partial tear.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A method of repairing a partial articular-sided rotator cuff tear of the tendon of a patient’s shoulder comprising the steps of:
establishing arthroscopic access to a glenohumeral joint space;
introducing a regenerative biological construct into the glenohumeral joint space;
advancing the biological construct to an articular side of the partially torn rotator cuff tendon;
positioning the biological construct against the partial rotator cuff tear;
passing one or more fixation sutures through the tendon;
securing the one or more fixation sutures on the bursal side of the torn tendon with the biological construct compressed to the articular side of the torn tendon;
tensioning the one or more fixation sutures from the bursal side to compress the biological construct against the articular side of the tendon and tying off the fixture sutures to the bursal side of the torn tendon; and
repairing the torn tendon while maintaining the tendon as a partial articular-sided rotator cuff tear without completing the tear into a full thickness tear.

2. The method of claim 1, where in the biological construct is positioned against the articular side of the tendon without accessing the tear from the bursal side.

3. The method of claim 1, wherein the biological construct remains positioned on the articular side of the tendon after the one or more fixations sutures are secured.

4. The method of claim 1, wherein no portion of the tendon is surgically severed to convert the partial articular-sided tear into a full-thickness tear during placement and fixation of the biological construct.

5. The method of claim 1, wherein the step of establishing an arthroscopic workspace includes creating an anterior portal through a rotator cuff interval of the should and also establishing a separate lateral portal within the shoulder positioned opposite the articular side of the partially torn rotator cuff.

6. The method of claim 5 wherein step of passing one or more fixation sutures through the tendon further includes inserting a spinal needle through the lateral portal and introducing at least two traction sutures through a rotator cuff of the patient to the bursal side of the tear.

7. The method of claim 6 wherein step of tensioning the one or more fixation sutures from the bursal side to compress the biological construct against the articular side of the tendon further includes tying two suture tails contained on the biological construct to the fixation sutures.

8. The method of claim 1, wherein the biological construct is introduced through an anterior portal into the glenohumeral joint space.

9. The method of claim 1, wherein the biological construct is advanced from a first rolled configuration to a second unrolled configuration after advancing through the glenohumeral joint space.

10. The method of claim 1, wherein advancing the biological construct is performed by pushing the biological construct with an inserter while simultaneously pulling one or more sutures attached to the biological construct.

11. The method of claim 1, wherein the biological construct spans substantially the entire partial articular-sided tear.

12. The method of claim 1 further comprising the step of securing one or more fixation sutures to a pledget positioned on the bursal side of the tendon.

13. The method of claim 1, further including the step of removing the traction sutures after the biological construct is positioned and prior to tying the suture tails.
